(19) United States
(12) Patent Application Publication
(10) Pub. No.: US 2008/0051902 A1
(43) Pub. Date: Feb. 28, 2008

(54) MODULAR INTERVERTEBRAL DISC PROSTHESIS AND METHOD OF REPLACING AN INTERVERTEBRAL DISC

(76) Inventor: James DWYER, Nashanic Station, NJ (US)

Correspondence Address: KENYON & KENYON LLP, 1500 K STREET N.W., SUITE 700, WASHINGTON, DC, 20005, US

(21) Appl. No.: 11/835,756

(22) Filed: Aug. 8, 2007

Related U.S. Application Data
(60) Provisional application No. 60/836,680, filed on Aug. 10, 2006.
(60) Provisional application No. 60/849,773, filed on Oct. 6, 2006.

(51) Int. Cl. A61F 2/44; A61B 17/56

(52) U.S. Cl. 623/17.16; 606/72; 623/17.15

(57) ABSTRACT

An intervertebral disc prosthesis is provided. The prosthesis includes a first and second plate containing modular anchors that are moveable from a radially un-extended position in a non-deployed configuration to a radially extended position in a deployed configuration. A method of replacing a natural intervertebral disc with a prosthetic disc is also provided.

### CROSS-REFERENCE TO RELATED APPLICATIONS

The present application claims priority to U.S. Provisional Application No. 60/836,680 filed on Aug. 10, 2006 and U.S. Provisional Application No. 60/849,773 filed on Oct. 6, 2006, both of which are incorporated by reference herein.

### FIELD OF THE INVENTION

The present invention relates to intervertebral disc prostheses and methods of replacing a natural intervertebral disc.

### BACKGROUND OF THE INVENTION

The intervertebral disc provides a mechanical cushion between adjacent vertebral segments of the spinal column and also maintains the proper anatomical separation between two adjacent vertebrae. This separation is necessary for allowing both afferent and efferent nerves to respectively exit and enter the spinal column. In some instances, genetic or developmental irregularities, trauma, chronic stress or degenerative disease can result in spinal pathologies necessitating removal of an intervertebral disc.

One option after removal of the intervertebral disc is completely replacing the disc with an artificial disc prosthesis. There are several designs of such total disc replacement prostheses, both for lumbar and cervical discs. One type of lumbar disc replacement consists of an upper metal plate, a lower metal plate and a polyethylene spacer positioned therebetween. The upper and lower plates have opposing rows of pegs on respective superior and inferior surfaces for attaching to bone. With this design, however, the fixation between the metal plates and the bone is inadequate as the small pegs do not sufficiently anchor the implant into the vertebral bodies. Another lumber disc replacement consists of a semi-constrained device including two metal endplates, each having a midline keel on an outer surface thereof. Such devices include metal-on-metal design or designs where a polyethylene core is positioned between the two metal endplates. In either design, although the midline keels on the metal endplates are powerful stabilizers and provide acute bone fixation, the keels may be difficult to remove if the implant needs revision. For example, such a design could require a complete corpectomy with a two-level spinal fusion if a revision is required.

Accordingly, there is a need for a modular interverterbral disc prosthesis that provides adequate fixation in adjacent bone and which allows for sufficient adjustability.

### SUMMARY OF THE INVENTION

In one embodiment, the present invention provides an intervertebral disc prosthesis member comprising a plate having a channel extending at least partially therethrough, a superior surface comprising a slot in fluid communication with the channel, and an inferior surface. The disc prosthesis further comprises a moveable anchor that is disposable in the channel.

In another embodiment, the present invention provides an intervertebral disc prosthesis that includes a first plate and a second plate. The first plate has a first channel extending at least partially therethrough, a superior surface comprising a slot in fluid communication with the first channel, and an inferior surface. A first moveable anchor is disposable in the first channel. The second plate has a second channel extending at least partially therethrough, an inferior surface comprising a slot in fluid communication with the second channel, and a superior surface. The disc prosthesis further includes a second moveable anchor that is disposable in the second channel. The disc prosthesis can further include a spacer positionable between the first and second plate.

The present invention also provides a method of replacing a natural intervertebral disc in a patient with a prosthetic intervertebral disc. The method utilizes a prosthetic intervertebral disc comprising a modular anchor, which is moveable from a radially un-extended position in a non-deployed configuration to a radially extended position in a deployed configuration. The method comprises removing a natural intervertebral disc from the intervertebral space and inserting a prosthetic intervertebral disc in a non-deployed configuration into the intervertebral space. The anchor is then deployed to extend into adjacent vertebral bodies and secure the prosthetic intervertebral disc in the intervertebral space.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is an enlarged perspective view of a disc prosthesis according to an embodiment of the present invention.

FIG. 2 is a sectional view of a disc prosthesis in a non-deployed configuration inserted between adjacent vertebrae.

FIG. 3 is a sectional view of the disc prosthesis of FIG. 2 in a deployed configuration.

FIG. 4 is an enlarged side view of an anchor of a disc prosthesis according to an embodiment of the present invention.

FIG. 5 is an enlarged side view of an anchor of a disc prosthesis according to another embodiment of the present invention.

FIG. 6 is an enlarged end view of a disc prosthesis according to an embodiment of the present invention.

FIG. 7 is an enlarged sectional view of a disc prosthesis according to an embodiment of the present invention.

FIG. 8 is an enlarged side view of a pin of a disc prosthesis according to an embodiment of the present invention.

FIG. 9 is an enlarged side view of a pin of a disc prosthesis according to an alternative embodiment of the present invention.

FIG. 10 is an enlarged perspective view of a disc prosthesis according to an embodiment of the present invention.

FIG. 11 is an enlarged perspective view of a disc prosthesis according to an alternative embodiment of the present invention.

FIG. 12 is a perspective view of a disc prosthesis according to an alternative embodiment of the present invention.

FIG. 13 is a perspective view of a disc prosthesis according to an alternative embodiment of the present invention.

FIG. 14 is a perspective view of a disc prosthesis according to an alternative embodiment of the present invention.

FIG. 15 is an enlarged side view of a disc prosthesis of the present invention according to an alternative embodiment of the present invention.

FIG. 16 is a side view of a spacer and a perspective view of an endplate of a disc prosthesis of the present invention according to an alternative embodiment of the present invention.

FIG. 16A is an exploded view of a disc prosthesis of the present invention according to an alternative embodiment of the present invention.

FIG. 17 is a perspective exploded view of a spacer and second plate of a disc prosthesis according to an alternative embodiment of the present invention.

FIG. 18A is a perspective view of an embodiment of a interbody fusion cage of a spinal kit according to an embodiment of the present invention.

FIG. 18B is a perspective view of another embodiment of an interbody fusion cage of a spinal kit according to an embodiment of the present invention.

FIG. 19 is a plan view of an instrument according to an embodiment of the present invention.

FIG. 20 is a perspective view of an alignment jig according to an embodiment of the present invention.

FIG. 21 is a sectional view of the instrument of FIG. 19 passing through the alignment jig of FIG. 20 to access an anchor of a plate of a disc prosthesis of the present invention.

### DETAILED DESCRIPTION OF THE INVENTION

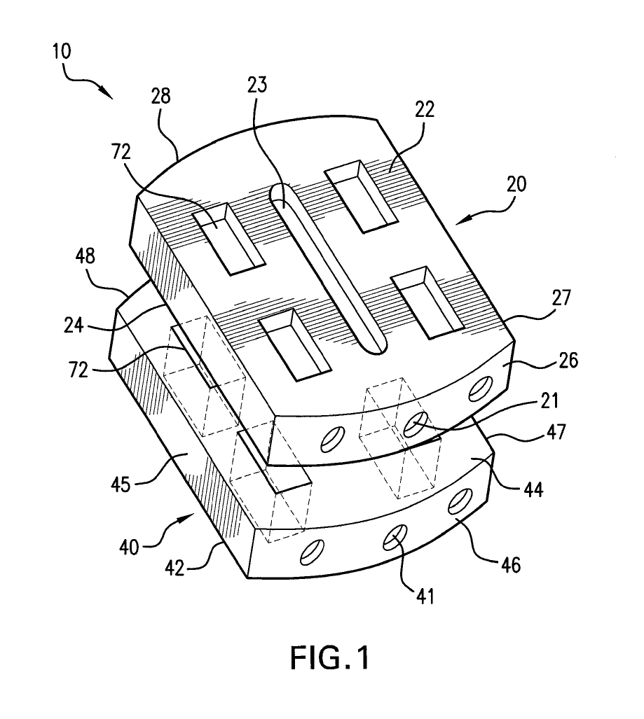

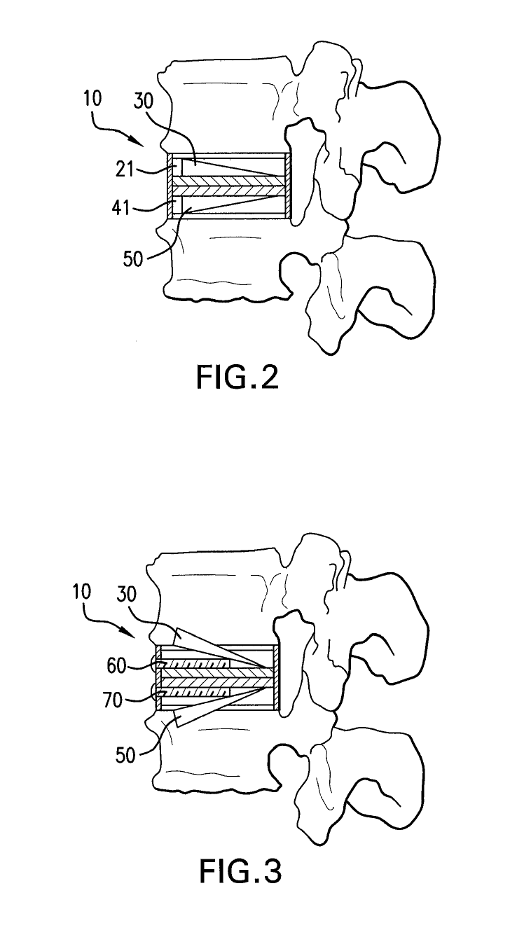

Referring to FIG. 1, in certain embodiments, the present invention provides an intervertebral disc prosthesis 10 comprising a first plate 20 and a second plate 40. First plate 20 has an upper surface 22, a lower surface 24, and side surfaces 25 to 28. As seen in FIG. 1, a first channel 21 extends at least partially through first plate 20. Upper surface 22 contains a slot 23, which is in fluid communication with first channel 21. Referring to FIG. 2, a first moveable anchor 30 is disposable in first channel 21. Similarly, referring back to FIG. 1, second plate 40 has a lower surface 42, an upper surface 44, and side surfaces 45 to 48. A second channel 41 extends at least partially through second plate 40. Lower surface 42 defines a slot 43 (not shown) in fluid communication with second channel 41. Similar to first plate 20, a second moveable anchor 50 is disposable in second channel 41, as shown in FIG. 2.

Referring to FIG. 2, in a non-deployed configuration, first and second anchors 30 and 50 remain substantially contained within respective channels 21 and 41, which can extend substantially the entire length between opposing side surfaces of respective first and second plates 20 and 40 or a distance less than substantially the entire length. In a preferred use, first and second plates 20 and 40 are inserted into an intervertebral space in a non-deployed configuration. With anchors 30 and 50 non-deployed, disc prosthesis 10 can be properly adjusted and aligned in the intervertebral space. Once the proper position of disc prosthesis 10 is obtained, first and second anchors 30 and 50 can be deployed. Referring to FIG. 3, in a deployed configuration, a portion of first and second anchors 30 and 50 protrude from respective slots 23 and 43, which can extend substantially the entire length between opposing side surfaces of first and second plate 20 and 40 or a distance less than substantially the entire length. In a deployed configuration, first and second anchors 30 and 50 extend into the underside and upperside, respectively, of the upper and lower vertebral body adjacent to disc prosthesis 10 to anchor first and second plates 20 and 40 thereto. As seen in FIG. 3, a portion of first and second anchor 30 and 50 can be urged out of respective channel 30 and 50 by a respective pin 60 and 70 that is inserted into the respective channel of first and second plate 20 and 40. Pin 60 and/or 70 can remain in the respective channel after deployment of first and second anchor 30 and 50 or can be removed.

The anchors of a disc prosthesis of the present invention allow for initial stability and acute fixation of the disc prosthesis in the intervertebral space. The moveability of the anchors allows the disc prosthesis to be repositioned or removed, both before and after the disc prosthesis has been secured in the intervertebral space. For example, before the disc prosthesis has been secured, it can be inserted within the intervertebral space and placed in the proper position with the anchors non-deployed. Once the proper position of the disc prosthesis has been determined, the anchors can be deployed and secured in the endplates of the vertebral bodies above and below the removed disc. If desired, the position of the disc prosthesis can be changed after it has been secured within the intervertebral space, since the anchors can be lowered or raised into the channels of the first and second plate, respectively, and the disc prosthesis can thus be re-positioned or removed.

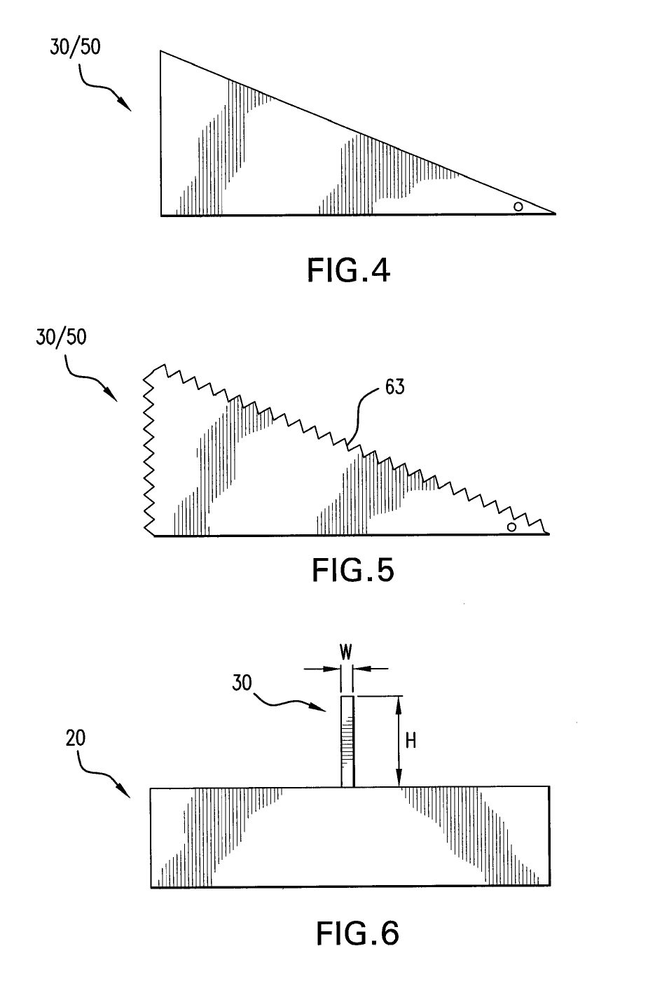

The anchors can have any suitable configuration that allows them to achieve purchase into the endplates of the vertebral bodies. For example, in the embodiment shown in FIG. 4, anchor 30/50 has a substantially smooth surface whereas in the embodiment shown in FIG. 5, anchor 30/50 has zigzag edges which comprise teeth 63. In a preferred embodiment, as illustrated in FIGS. 2 and 3, anchors 30 and 50 are wedge-shaped with the widest section of the anchor leading the protrusion from the respective slot of the respective plate and the narrowest section remaining in the respective channel in a deployed configuration. The anchors can have any length that permits a sufficient part of the anchors to protrude from the slots of the plates in a deployed configuration to secure the anchors in the vertebral bodies. Preferably, the part of each anchor that protrudes from the plate slot has a length that is greater than half of the overall length of the plate between opposing side surfaces. Also preferably, when fully deployed, the vertical height (H) of each anchor is greater than its width (W), as shown in FIG. 6. The anchors of the first and second plate can have the same or different configurations.

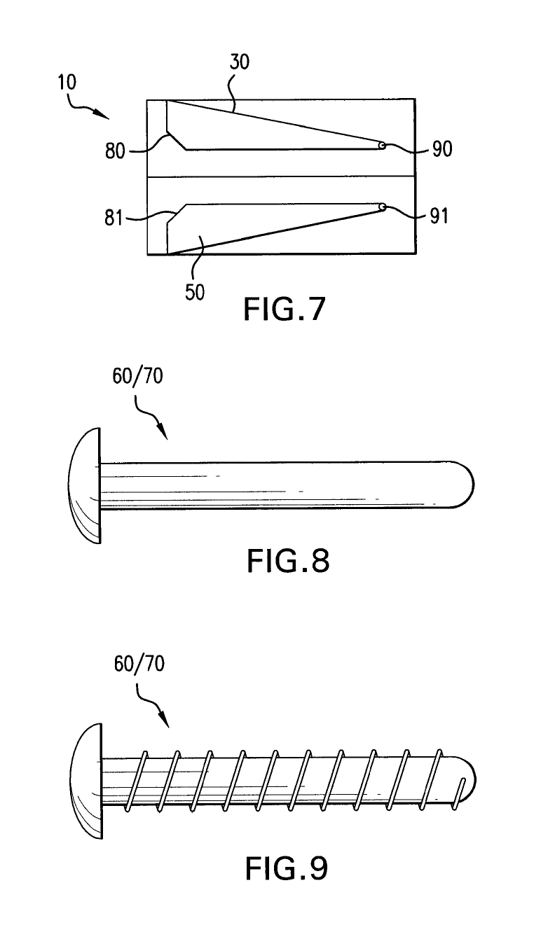

Referring to FIG. 7, anchor 30 and 50 can be pivotally connected to respective plates 20 and 40 via respective pivot pins 90 and 91. Anchors 30 and 50 can be pre-disposed in respective plates 20 and 40 or can be inserted into respective plates 20 and 40 during peri-operative or pre-operative assembly of disc prosthesis 10. Referring again to FIG. 7, anchors 30 and 50 can have an indented edge 80 and 81, respectively, that allow pin 60 and 70 to respectively access anchor 30 and 50 from underneath in order to raise and lower respective anchor 30 and 50. Referring to FIGS. 8 and 9, pin 60/70, can have a substantially smooth surface as illustrated in FIG. 8 or a threaded surface as illustrated in FIG. 9. Of course, in embodiments where pin 60/70 is threaded, the respective channel in which the pin is inserted is also cooperatively threaded to engage pin 60/70.

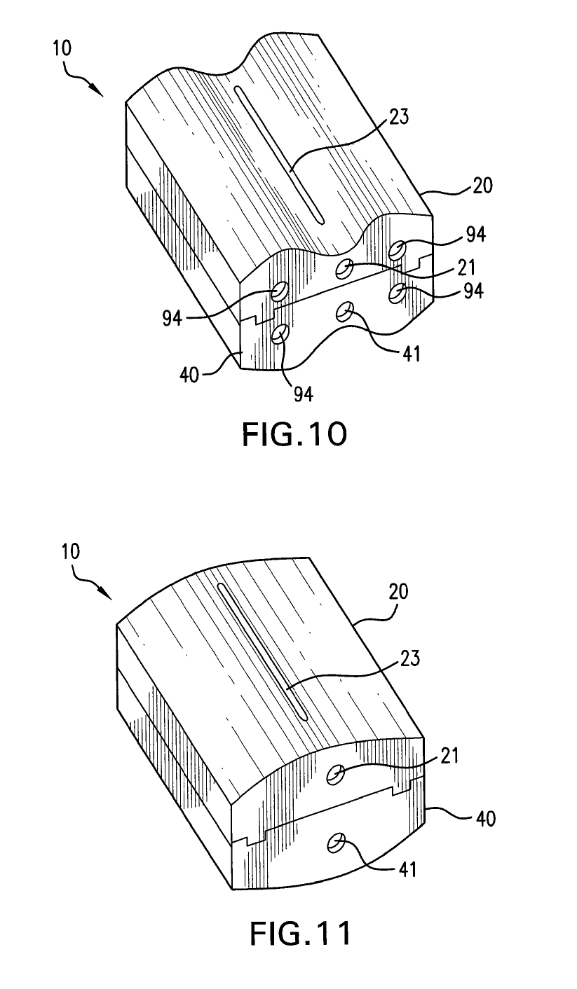

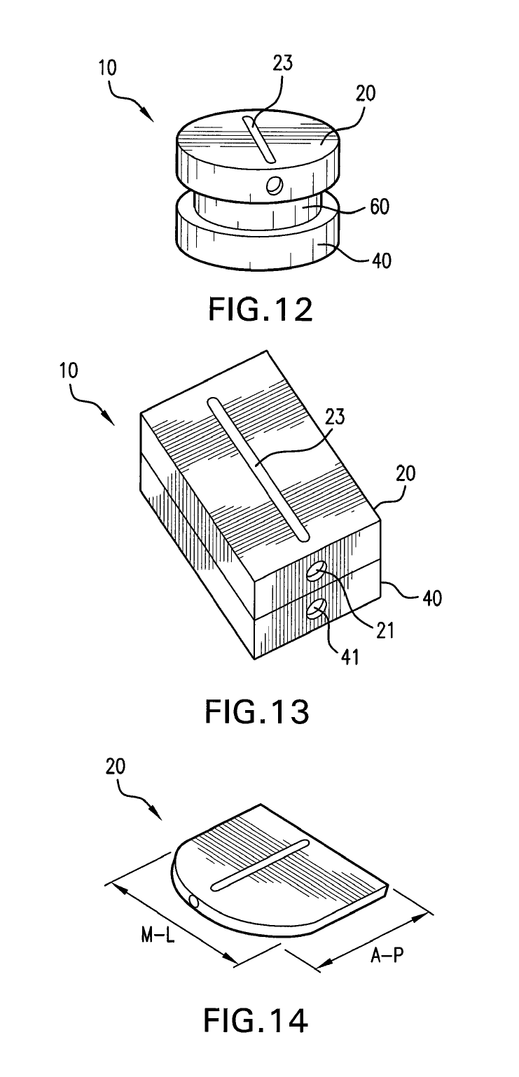

The plates, which carry the anchors of a disc prosthesis of the present invention, are adapted to replace the removed disc and can have any suitable configuration and size that allow the plates to fit within the intervertebral space at any spinal level. The plates can also match the shape and contour of the vertebral endplates against which they abut to better mate against the vertebral endplates. For example, the first plate can have an upper surface that allows for more optimal or extended surface area contact with the adjacent porous or cancellous interior surface of a prepared upper vertebral body and the second plate can have a lower surface that allows for similar contact with a lower vertebral body. Non-limiting examples of plate configurations include an arcuate profile, as shown in FIG.10, a domed or convex-like profile, as shown in FIG. 11, a cylindrical profile as shown in FIG. 12, or a rectangular profile as shown in FIG. 13. The configuration of the plates can also depend on the spinal level at which the prosthesis is being inserted. For example, the anterior-posterior (A-P) and medial-lateral (M-L) dimensions of the first and second plates can be chosen to suit typical lumbar/cervical disc dimensions, such as an A-P dimension of about 20 to 25 mm and a M-L of about 28 to 35 mm as viewed in illustrative FIG. 14. In the embodiment illustrated in FIG. 14, (in which only plate 20 is shown for purposes of clarity) plate 20 has a wedge-like shape such that one side of the plate has a height less than the height of the opposing side. Specifically, the plate can have a tapered thickness that increases in the anterior to posterior direction to provide an anterior to posterior lordotic taper to better restore the natural curvature of the spine. Various combinations of these profiles may be used as well, and the first and second plates can have the same or different configurations.

The plates and anchors of a disc prosthesis of the present invention can be fabricated from any suitable biocompatible sterile material such as a metallic material, a shape memory alloy, a ceramic material, a polymeric material, or any combination thereof. Non-limiting of metallic materials include metals and metal alloys, such as, for example, titanium, stainless steel, and cobalt chromium alloy including a cobalt chromium molybdenum alloy. Non-limiting examples of ceramic materials include zirconium oxide, aluminum oxide or sintered silicon nitride. Non-limiting examples of polymeric materials include polyarylesterketones including polyetheretherketone (PEEK) and polyetherketoneketone (PEKK). The polymer materials can also be reinforced with fillers, or fibers or oriented to provide additional mechanical properties. For example, the polymer material can be reinforced with bioceramic or bioglass particles such as, for example, hydroxyapatite, which also act as bioactive, bony ingrowth agents and provides a reservoir of calcium and phosphate ions.

Irrespective of the material from which the plates and anchors of disc prostheses of the present invention are fabricated, preferably the plates and/or anchors have a porous surface thereon to accommodate bone in-growth to provide solid fixation of the prostheses. In one embodiment, the upper surface of the first plate and the lower surface of the second plate include a porous coating or osteoconductive mesh structure. Alternatively, the surfaces can be made porous, such as by titanium plasma spray. For example, first and second plates can comprise a titanium bead coating applied onto their respective upper and lower surfaces via spraying or sintering. Alternatively, the outer surfaces of the plates can be roughened in order to promote bone in-growth into the defined roughened surfaces of the disc prosthesis. Referring to FIG. 1, in an alternative embodiment, plates 20/40 are fenestrated such that plates 20/40 defines pores 72 extending from the upper surface to the lower surfaces thereof. As described in more detail below, one use of such an embodiment is if it is desired to revise a disc prosthesis to an interbody fusion cage. In such embodiments, the pores preferably have a rectangular cross-sectional shape to facilitate bone growth should conversion to fusion be necessary.

The porous layer or surface on the first and/or second plate may also deliver desired pharmacological agents. The pharmacological agent may be, for example, a growth factor to assist in the repair of the endplates and/or the annulus fibrosis. Non-limiting examples of growth factors include a bone morphogenetic protein, transforming growth factor (TGF-β), insulin-like growth factor, platelet-derived growth factor, fibroblast growth factor or other similar growth factor or combinations thereof having the ability to repair the endplates and/or the annulus fibrosis of an intervertebral disc.

In other embodiments of the invention, the pharmacological agent may be one used for treating various spinal conditions, including, for example, degenerative disc disease, spinal arthritis, spinal infection, spinal tumor and osteoporosis. Such agents include, for example, antibiotics, analgesics, anti-inflammatory drugs, including steroids, and combinations thereof.

Referring back to FIG. 12, in an embodiment, a disc prosthesis 10 of the present invention further comprises a spacer 60 configured to separate and fit between first plate 20 and second plate 40. The spacer can be of any configuration suitable to achieve this purpose and can be fabricated from any suitable biocompatible material. Non-limiting examples of such materials include plastic materials, such as polyethylene, including a ultra high molecular weight cross-linked polyethylene, polymethacrylate, polyurethane, durometer, a hydrogel, or combinations thereof. In an embodiment, the spacer is fabricated from a material that has elastic properties substantially equivalent to the natural elastic properties of the human body's intervertebral disc.

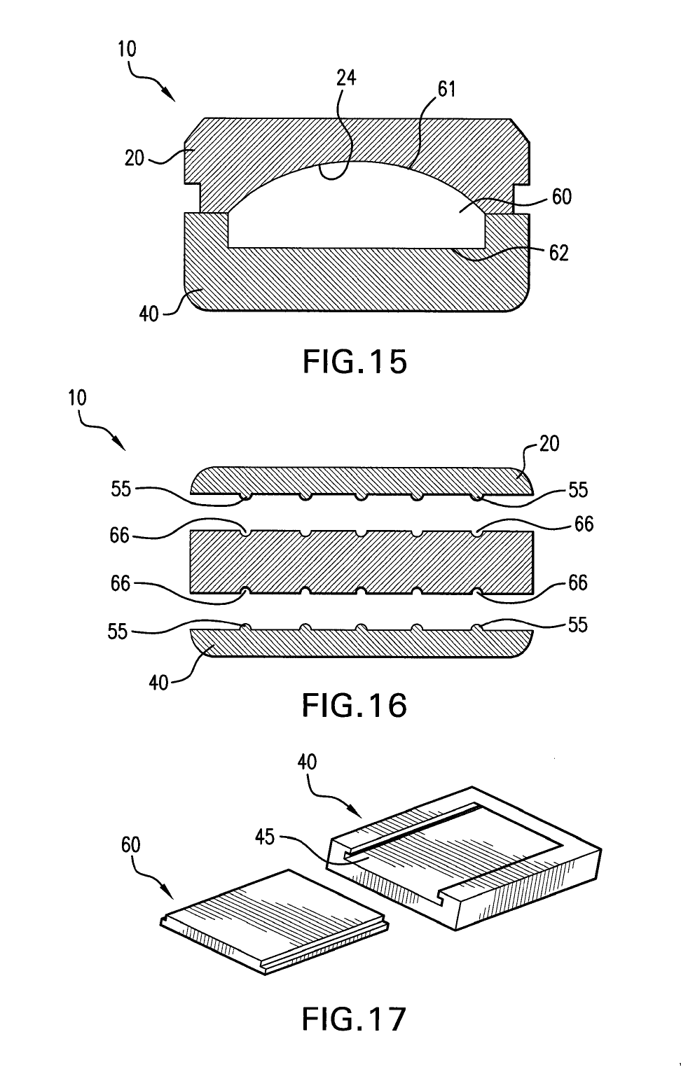

In certain embodiments, the plates and spacer can be configured to have articulating surfaces to facilitate pivotal and/or rotational movement of the first and second plates relative to one another. For example, the spacer can have a convex top surface articulating with a concave lower surface of the first plate. Specifically, referring to FIG. 15, spacer 60 has a spherically upward-curved top surface 61 and a substantially flat bottom surface 62. The spherical top surface 61 dips in complementary fashion into the spherically curved indentation 24 on the lower surface of first plate 20, where it forms a ball joint, which enables a certain pivotibility of the first plate relative to the second plate. In embodiments where no spacer is used, the first and second plates can articulate with one another. For example, first plate can have a concave female socket portion which articulates with a male concave ball portion on second plate or vice versa.

In certain embodiments, a disc prosthesis of the present invention is configured to maintain the range of motion provided by the spinal segments in which the disc is inserted. Further, in certain embodiments, the center of rotation of the prosthesis matches the normal central of rotation of the area of the spine in which the prosthesis is implanted to decrease the load on the facet joints. For example, if the prosthesis is implanted between lumber vertebrae, the prosthesis can have a center of rotation located posteriorly, for example at 65% of the length between the anterior end and posterior end.

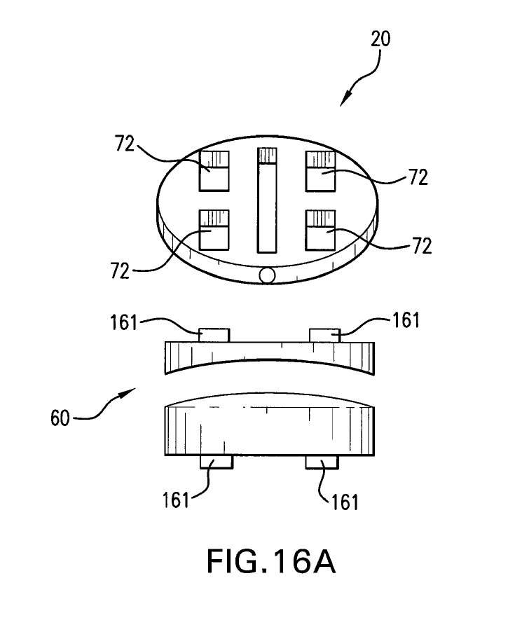

The plates and spacers (in embodiments including a spacer) can include connection mechanisms that secure the plates to the spacers or the plates to each other to prevent undesired relative movement thereof. The various parts of a disc prosthesis can be connected via any connection mechanism known in the art, such as, for example, male/female engagement, interference fit, adhesion, threaded engagement, positive interlockment and connection mechanisms described in U.S. Pat. No. 6,726,720, which is incorporated by reference herein. For example, as shown in FIG. 16, first and second plate 20 and 40 comprise protrusion elements 55 that are adapted to be securely received by apertures 66 in spacer 60 to inhibit undesirable slidable movement of first and second plate relative to spacer 60. In embodiments where plates 20 and 40 define pores 72 extending from the upper to lower surfaces thereof, spacer 60 can includes protrusion elements 161 that are adapted to be securely received by pores 72 of first plate 20 and/or second plate 40, as shown in FIG. 16A. In FIG. 16A, spacer 60 is illustrated as a two-piece device. However, spacer 60 could also be a one-piece device that is a single solitary piece that does not have individual combinable components that can be assembled together into a single unit. Referring to FIG. 17, in other embodiments, second plate 40 can define a central indentation 45 which receives spacer 60 such that spacer 60 snap fits into second plate 40.

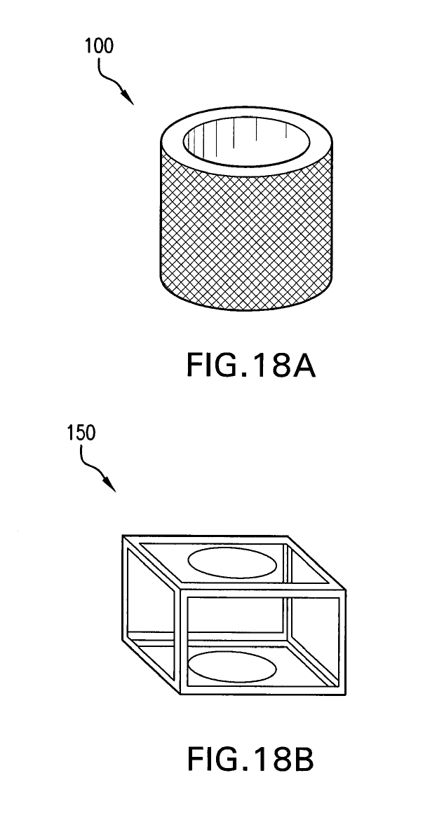

As mentioned above, in certain embodiments, plates 20/40 of disc prosthesis 10 can be fenestrated, if it is desired, for example, to revise disc prosthesis 10 to an interbody fusion cage. In such embodiments, the present invention provides a kit comprising such a disc prosthesis and at least one, and preferably two, interbody fusion cages. The spinal fusion cage can be inserted between the first and the second plate (to replace the spacer in embodiments comprising a spacer). The interbody fusion cage can be any type known in the art such as, for example, a vertical fusion cage 100, as shown in FIG. 18A, or a rectangular fusion cage 150, as shown in FIG. 18B. A non-limiting example of a vertical fusion cage is a Harms cage and a non-limiting example of a rectangular fusion cage is a Brantigan cage. The first and/or second plate can define a ring or depression (similar to the indentation 45 of FIG. 17) to accept a fusion cage of the same dimensions as the ring or depression. Such a ring or depression can allow the cage to lock or interface with the first and/or second plates.

In certain embodiments, the present invention also provides a kit with multiple disc prostheses (including multiple spacers) with varying heights and lordotic angles to restore a unique individual anatomy. The kit can include plates with different standardized A-P depths, M-L widths and spacers with varying heights to accommodate the physiological range of intervertebral spaces. With respect to inserting an intervertebral disc prosthesis of the present invention, a variety of tools can be used to separate the adjacent vertebrae, position the plates and insert the spacer or insert the pre-constructed disc prosthesis into the intervertebral space. Thus, a disc prosthesis of the present invention can include features which permit the disc prosthesis to be used in connection with an insertion tool. For example, referring to FIG. 10, first and second plates 20 and 40 can define apertures 94 on side surfaces thereof which are adapted to receive an insertion tool that can grasp the plates to move the disc prosthesis 10 along a path in the insertion direction (outside patient to inside patient).

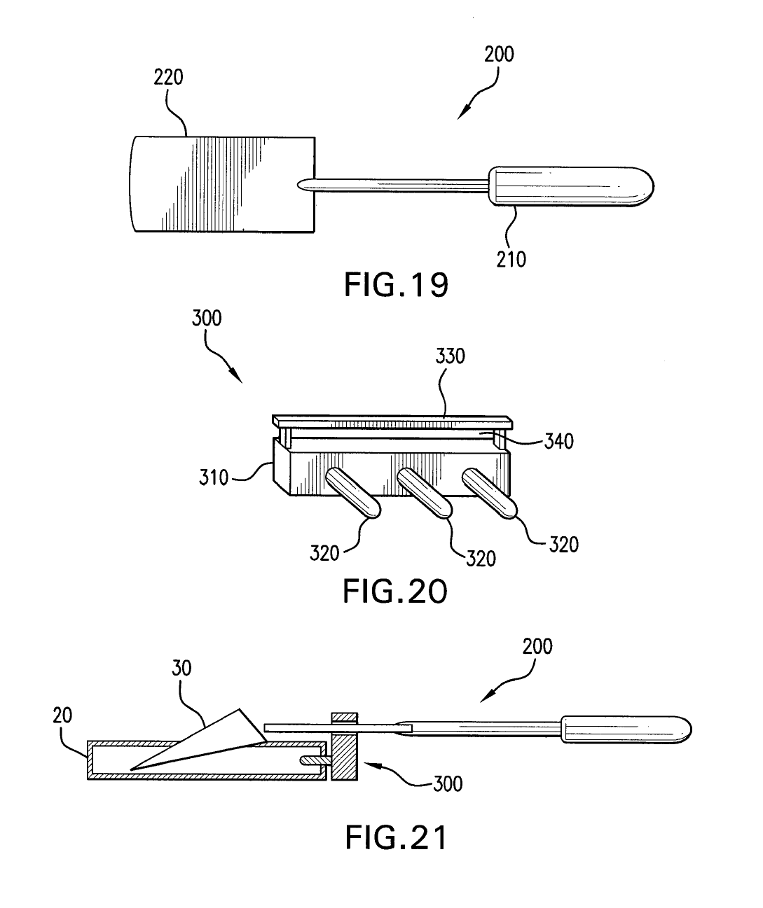

In certain embodiments, the present invention provides a kit with instrumentality to assist in re-positioning or removing a disc prosthesis after implantation thereof. Such instrumentality can comprise, for example, an instrument to urge the anchors of a disc prosthesis into a non-deployed configuration. For example, referring to FIG. 19, in an embodiment, such an instrument can comprise a paddle 200 having a handle 210 at a proximal portion thereof and a flattened portion 220 at a distal portion thereof. To re-position an anchor after disc prosthesis is implanted, paddle 200 can be inserted in the intervertebral space and impact against the anchor to seat the anchor substantially within the respective channel of the respective plate. With the anchor no longer secured in vertebrae, the respective plate can be removed or re-positioned as desired. Further, if both anchors are disengaged from vertebrae, the disc prosthesis can be removed or re-positioned. Referring to FIG. 20, in certain embodiments, the instrumentality can further comprise an alignment jig 300 comprising a base 310 having a plurality of pegs 320 extending therefrom that can be inserted in the channel of a plate of disc prosthesis and any insertion apertures, to removably attach jig 300 to a plate of a disc prosthesis. Jig 300 can further comprise a guide member 330 attached to base 310 defining a passage 340 through which flattened portion 220 of paddle 200 passes, as shown in FIG. 21. Guide member 330 guides flattened portion 220 in a direction substantially parallel to the insertion plane such that flattened portion 220 can access and impact against the anchor. Of course other configurations of instruments and alignment jigs can be used to assist in re-positioning or removing a disc prosthesis after implantation thereof.

A disc prosthesis of the present invention can be implanted via an anterior, posterior, lateral, or extreme lateral approach and the present invention contemplates embodiments of a disc prosthesis with anchors oriented accordingly. A non-limiting example of a process for inserting a prosthesis of the present invention will now be provided. The patient is placed in supine position on a standard radiolucent operative table. The surgical approach is anterior retroperitoneal. Once the appropriate disc level is exposed, a complete dicectomy is performed including removal of the cartilage from the superior and inferior endplates and removed of the posterior longitudinal ligament. Next, the disc prosthesis, pre-assembled on a back table, is inserted into the intervertebral space with the anchors non-deployed. Under fluoroscopic guidance, the prosthesis is placed in the midline in the frontal plane as posteriorly as possible in the sagittal plane without entering the spinal canal. Distraction is next performed with a distractor instrument and the spacer is inserted between the first and the second plate. The anchors are then deployed to secure the prosthesis in the intervertebral space. Finally, the insertion instruments are removed and water tight closure is performed. If replacing a lumbar disc, preferably, the prosthesis is inserted through a minimal incision through the lumbar spine, typically a mini-retroperitoneal approach.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

I claim:

1. An intervertebral disc prosthesis comprising:
a first plate having a first channel extending at least partially therethrough, an upper surface containing a slot in fluid communication with the first channel, and a lower surface;
a first moveable anchor disposable in the first channel;
a second plate having a second channel extending at least partially therethrough, a lower surface containing a slot in fluid communication with the second channel, and an upper surface; and
a second moveable anchor disposable in the second channel.

2. The intervertebral disc prosthesis of claim 1, wherein the first and second anchors are disposed in the first and second channels, respectively.

3. The intervertebral disc prosthesis of claim 1, wherein the first and second anchors have substantially smooth surfaces.

4. The intervertebral disc prosthesis of claim 1, wherein the first and second anchor are each wedge-shaped.

5. The intervertebral disc prosthesis of claim 1, wherein the first and second anchors have zigzag edges comprising teeth.

6. The intervertebral disc prosthesis of claim 1, wherein the upper surface of the first plate and the lower surface of the second plate have convex configurations.

7. The intervertebral disc prosthesis of claim 1, wherein the first and second plate are wedge-shaped.

8. The intervertebral disc prosthesis of claim 1, wherein the first plate defines pores extending from the upper surface to the lower surface thereof and the second plate defines pores extending from the upper surface to the lower surface thereof.

9. The intervertebral disc prosthesis of claim 1, wherein the lower surface of the first plate is concave and the upper surface of the second plate is convex.

10. The intervertebral disc prosthesis of claim 1, further comprising a spacer positionable between the first and second plate.

11. The intervertebral disc prosthesis of claim 10, wherein the lower surface of the first plate is concave and an upper surface of the spacer is convex.

12. The intervertebral disc prosthesis of claim 9, wherein the first plate comprises connection means and the spacer comprises reciprocal connection means that accept the connection means of the first plate to secure the first plate to the spacer.

13. The intervertebral disc prosthesis of claim 9, wherein the second plate defines a central indentation which receives the spacer.

14. The intervertebral disc prosthesis of claim 1, further comprising a first pin insertable in the first channel and a second pin insertable in the second channel.

15. The intervertebral disc prosthesis of claim 14, wherein each of the first and second pins have substantially smooth surfaces.

16. The intervertebral disc prosthesis of claim 14, wherein each of the first and second channels are threaded and each of the first and second pins are cooperatively threaded.

17. The intervertebral disc prosthesis of claim 1, wherein the first plate further comprises a porous layer coating the upper surface thereof and the second plate further comprises a porous layer coating the lower surface thereof.

18. The intervertebral disc prosthesis of claim 1, wherein the first and second plate define apertures on a side surface thereof that are adapted to receive an insertion tool.

18. An intervertebral disc prosthesis member comprising:
a plate having a channel extending at least partially therethrough, an upper surface comprising a slot in fluid communication with the channel, and a lower surface; and
a moveable anchor disposable in the channel.

19. The intervertebral disc prosthesis of claim 1, wherein the moveable anchor is disposed in the channel.

20. A spinal kit comprising:
a plurality of disc prostheses, each of the disc prostheses comprising:
a first plate having a first channel extending at least partially therethrough, an
upper surface comprising a slot in fluid communication with the first channel, and a lower surface;
a first moveable anchor disposable in the first channel;
a second plate having a second channel extending at least partially therethrough, a lower surface comprising a slot in fluid communication with the second channel, and an upper surface; and
a second moveable anchor disposable in the second channel, wherein each of the plurality of disc prostheses have different anterior-posterior depths and/or different medial-lateral widths.

21. A spinal kit comprising
the disc prosthesis of claim 1; and
an interbody fusion cage comprising a hollow cylindrical body having an outer wall defining a plurality of fenestrations.

22. The spinal kit of claim 21, wherein the outer wall of the interbody fusion cage is at least partially threaded.

23. A spinal kit comprising:
The disc prosthesis of claim 1;
a paddle comprising a handle at a proximal portion thereof and a flattened portion at a distal portion thereof; and
an alignment jig.

24. A method of replacing a natural intervertebral disc in a patient with a prosthetic intervertebral disc comprising:
providing a prosthetic intervertebral disc comprising a modular anchor moveable from a radially un-extended position in a non-deployed configuration to a radially extended position in a deployed configuration;
removing a natural intervertebral disc from the intervertebral space;
inserting a prosthetic intervertebral disc in a non-deployed configuration in the intervertebral space; and
deployed the anchor to extend into adjacent vertebral bodies and secure the prosthetic intervertebral disc in the intervertebral space.
